(19) United States
(12) Patent Application Publication
(10) Pub. No.: US 2011/0237906 A1
(43) Pub. Date: Sep. 29, 2011

(54) SYSTEM AND METHOD FOR GRAPHICAL DISPLAY OF MEDICAL INFORMATION

(75) Inventor: Serguei Kabakov, Laurel, MD (US)

(73) Assignee: GENERAL ELECTRIC COMPANY, Schenectady, NY (US)

(21) Appl. No.: 12/732,724

(22) Filed: Mar. 26, 2010

(51) Int. Cl. A61B 5/00; G06T 11/20

(52) U.S. Cl. 600/301; 345/440; 345/440.1

(57) ABSTRACT

In one embodiment, an apparatus for graphical display of medical information is provided. The apparatus comprises a memory configured to store medical information indicative of multiple categories, each category of medical information representing information about plurality of physiological variables, a processing system configured to determine a data value for at least one physiological variable in each of the category of medical information and further configured for generating a color code using the data values and a display system for displaying a color coded image comprising multiple sections, each section displaying color codes associated with a single physiological variable.

### FIELD OF INVENTION

The invention relates to patient evaluation systems and methods, and more particularly to graphical methods and systems for rapidly conveying medical information.

### BACKGROUND OF THE INVENTION

Medical patient monitors are typically employed by physicians and other health care providers for monitoring patients in the operating room, intensive care units and emergency rooms. Monitors used in operating rooms may, for example, display such variables as blood oxygen saturation in the arterial and venous vessels, cardiac output, heart rate, mean arterial pressure, central venous pressure, arterial systolic pressure, arterial diastolic pressure, tracheal gas flow, ventilator pressure, and the volume percentage of oxygen and CO.sub.2 in the patient's mouth. An array of sensors are connected to the patient for acquiring such data which is displayed on the screen of a monitor either in graphic or numeric form. Such data may also be recorded or displayed on analog or digital strip chart recorders.

In prior art patient monitoring systems, it was necessary for the user to scan through and evaluate a great deal of data including various graphic and/or numeric displays in order to monitor the patient's condition, and to determine the occurrence of significant events which may require immediate attention and to quickly establish a priority of action.

These prior art monitoring systems and methods do a poor job of highlighting the most relevant or important information when the information is conveyed, increasing the risk of missing critical information that could be used to diagnose, treat, or otherwise assist a patient or doctor.

Another limitation associated with the prior art monitoring systems is that the medical professionals differ greatly in how they interpret displayed variables. For example, in a study, while using an electronic fetal monitoring system, four obstetricians examined fifty fetal heart tracings, and they agreed in twenty two percent of the cases. Two months later, the same four obstetricians re-evaluated the same fifty tracings and changed their interpretations on nearly one of every five.

To alleviate this problem, American College of Obstetricians and Gynecologists published new fetal monitoring guidelines implying that medical professionals need to look at entire clinical picture, which is supposed to include not only FHR (Fetal Heart Rate) and UA (Uterine Activity) waveforms but also maternal vital signs as well as medications taken by patient.

Unfortunately, conventional strip chart recording when FHR and UA waveforms are printed on the paper at speed, say, 3 cm/minute is not compliant with new guidelines. the reasons for this include the time period for labor and delivery and limited nature of strip chart recording. Firstly, labor and delivery may last on an average up to eight hours and therefore it is hard for medical professionals to look at entire clinical picture unrolled along fifteen meters of recording. Secondly, format of strip chart recording covers neither maternal vital signs nor medications taken by the patient

Therefore, it will be appreciated that physicians, nurses, administrators, and other medical professionals, as well as patients and other laypersons may find difficulty in reviewing, digesting and understanding the information presented in conventional fashion, and especially in discerning the most important information contained in displays using conventional methods and systems.

Hence, there exists a need to provide a system and method of displaying medical information that facilitates medical professionals in reviewing and understanding the information presented and is also capable of presenting a large amount of data in a limited space.

### BRIEF DESCRIPTION OF THE INVENTION

The above-mentioned shortcomings, disadvantages and problems are addressed herein which will be understood by reading and understanding the following specification.

In one embodiment, an apparatus for graphical display of medical information is provided. The apparatus comprises a memory configured to store medical information indicative of multiple categories, each category of medical information representing information about plurality of physiological variables, a processing system configured to determine a data value for at least one physiological variable in each of the category of medical information and further configured for generating a color code using the data values and a display system for displaying a color coded image comprising multiple sections, each section displaying color codes associated with one of the physiological variables.

In another embodiment, a system for simultaneously displaying a plurality of physiological variables indicative of the condition of a patient is provided. The system comprises an acquisition system for receiving medical information indicative of multiple categories, each category of medical information comprising signals corresponding to one or more physiological variables, a processing system for generating a plurality of data values, each of which is indicative of one of the signals, the processing system further configured for converting each of the data values into color codes and a display system for separately and simultaneously displaying each of the color coded values in a graphical form in a time correlated relation wherein variations in the data values are emphasized by differences in color or shade.

In yet another embodiment, a method for generating color-coded image of medical information is provided. The method comprises steps of obtaining one or more data values for a physiological variable representing medical information, grouping the data values into one or more data ranges, the data range representing a range of data values, mapping colors to data ranges in a manner that allows the colors to visually indicate the magnitude of the data ranges and marking an indicator for each data value on a graph, the indicator marked with the mapped color of the data range that comprises the data value of the physiological variable.

In yet another embodiment, a computer program product for implementing within a computer system a method for representing patient medical information as a colorgram is provided. The computer program product comprises a computer readable medium for providing computer program code means utilized to implement the method. Further, the computer program code means is comprised of executable code for implementing steps for receiving one or more data values for a physiological variable representing medical information, comparing each data value to a color map in which different color shades are designated for numeric values of the physiological variable, determining a color code for each data value and displaying color coded image, the color coded image comprising a sequence of color codes representing various data values of the physiological variable.

Systems and methods of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 shows a block diagram of an apparatus for graphical display of medical information in accordance with one embodiment;

FIG. 2 shows a table representing various color maps that are used in generating color-coded image of medical information, as described in one embodiment;

FIG. 3 shows a color table for fetal heart rate (FHR) waveform, as described in one embodiment;

FIG. 4 shows a flow diagram depicting a method for generating color-coded image of medical information, as described in one embodiment;

FIG. 5 shows a block diagram of a system for conveying medical information in a color-coded image, in accordance with one embodiment;

FIG. 6 shows a color table illustrating the colors mapped for each physiological variable using jet colormap;

FIG. 7 shows a table providing clinical meaning for the colors mapped for each physiological variable using jet colormap;

FIG. 8 shows a macrocell representing spatial integration as described in one embodiment; and

FIG. 9 shows a colorgram with macrocell shown on FIG. 8, as described in one exemplary embodiment.

### DETAILED DESCRIPTION OF THE INVENTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

The invention provides systems and methods for converting medical information into color coded images that represent the entire clinical picture in a condensed colorful form.

Medical information, as used herein, includes any information that may be medically relevant, including symptom information, general patient information, medical history information, family medical history information, medicinal information, treatment information, dietary information, pharmaceutical information, or any other information that relates to an individual.

For simplification the invention is explained with reference to a maternal fetal monitoring system. However, skilled artisans shall appreciate that the embodiments of the invention can as well be applied to other patient monitoring systems. Further, with reference to the maternal fetal monitoring system any reference to patient may be comprehended as a pregnant woman. However, in general the word “patient” may be interpreted broadly to include any human being or animal undergoing medical diagnosis or treatment.

Further, the medical information with reference to the fetal monitoring system, includes several category of medical information. By way of example, the categories may include fetal vital signs, maternal vital signs, integrated vital signs, medications taken by the patient and physical procedures applied to the patient during labor and delivery. Further, each category of medical information may be represented by several physiological variables.

For example, the fetal vital sign category may comprise the following physiological variables, namely, FHR (fetal heart rate waveform), FHRad (fetal heart rate acceleration/deceleration waveform), LTV (long-term FHR variability waveform), STV (short-term FHR variability waveform), UA (uterine activity waveform), FMD (fetal movement detection waveform), SQD (signal quality detection waveform) and coincidence waveform (waveform that registers coincidence between fetal and maternal heart rates).

The maternal vital signs category may comprise the following physiological variables, Temp (temperature waveform), SPO2 (pulse oximeter oxygen saturation waveform), MHR (maternal heart rate waveform), RR (respiration rate waveform), sBP (systolic blood pressure waveform) and dBP (diastolic blood pressure waveform).

The integrated vital signs category represents integration between the fetal vital signs and the maternal vital signs and may comprise the following physiological variables, arithmetic integration waveform, logical integration waveform, temporal integration waveform and spatial integration waveform.

The medication category represents various medications taken by patient and therefore comprises the following physiological variables namely, oxytocin (to accelerate labor), tocolytic (to suppress premature labor) and IV fluids (to hydrate patient).

The physical procedure category represents physical procedures applied to the patient during labor and delivery. The physical procedure category comprises the following physiological variables namely, FSs (fetal stimulus by sound), FSve (fetal stimulus by vaginal exam), CMPss (change maternal position side-to-side), CMPt (change maternal position Trendelenburg) and CMPkc (change maternal position knee-chest).

While five categories of medical information have been illustrated along with corresponding physiological variables any number of categories and physiological variables may be utilized, so as to suit a particular diagnosis or treatment purpose.

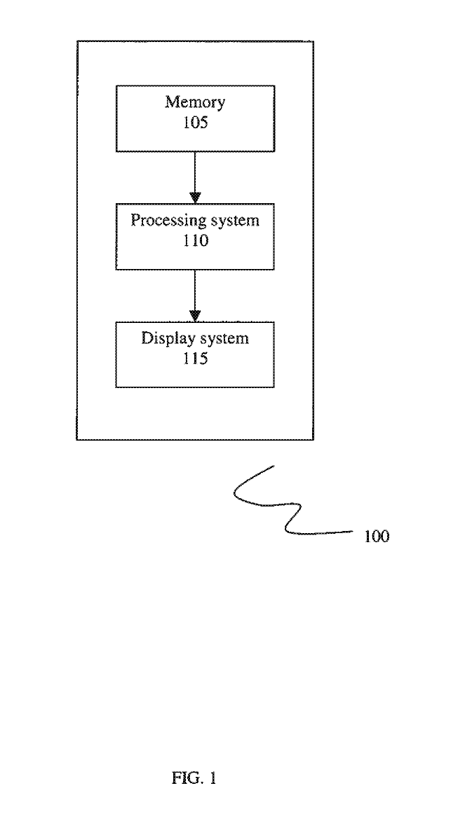

In one embodiment, an apparatus for graphical display of medical information is provided. The apparatus 100 shown in FIG. 1 comprises a memory 105 configured to store medical information indicative of multiple categories, each category of medical information representing information about plurality of physiological variables, a processing system 110 configured to determine a data value for at least one physiological variable in each of the category of medical information and further configured for generating a color code using the data values and a display system 115 for displaying a color coded image comprising multiple sections, each section displaying color codes associated with one of the physiological variables.

A color is mapped to a data value by selecting the color from a range of colors where the individual colors within the range of colors transition in relationship to the transition in the magnitude of the data values, such that changes in the colors visually indicate changes in the data values.

The color codes for the data values corresponding to a single physiological variable are generated from a color map. The color map comprises a predetermined sequence of colors selected from visible spectrum consisting of the colors red, orange, yellow, green, blue, indigo and violet.

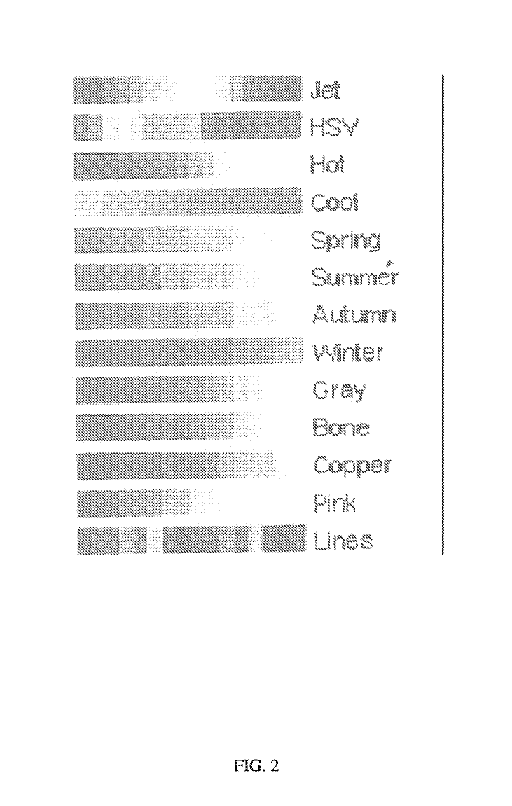

A typical color map may contain 256 squares, which may be any combination of color, shades of a single color or shades of gray. FIG. 2 shows examples of various color maps that can be used for this application. Though these color maps are shown as comprising the shades of gray, skilled artisans shall appreciate that each of these color maps may comprise multiple colors and/or shades of multiple colors where the variation in the color in each square corresponds to variations in the magnitude of the physiological variable.

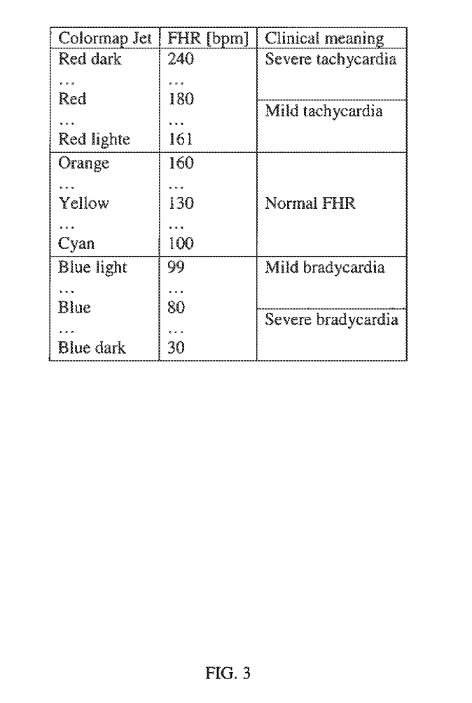

Each color in the color map is mapped to a single data value or a range of data values corresponding to a single physiological variable. A data range is a range of data values having an upper and a lower limit. The optimum data values in the example FHR physiological variable range from 100 beats per minute (BPM) to 160 BPM, and the physiological limits are 30 BPM on the lower end and 240 BPM on the upper end. To set up the color-mapped display for this particular variable, the range from 30 to 240 is divided into one or more number of equal or unequal segments, and each of these segments is mapped to a particular color. This is shown in the color table of FIG. 3. FIG. 3 also shows the color spectrum that is mapped to the various data value ranges.

Accordingly, in another embodiment, a method for generating color-coded image of medical information is provided. The method comprises obtaining one or more data values for a physiological variable representing medical information, grouping the data values into one or more data ranges, the data range representing a range of data values, mapping colors to data ranges in a manner that allows the colors to visually indicate the magnitude of the data ranges and marking an indicator for each data value on a graph, the indicator marked with the mapped color of the data range that comprises the data value of the physiological variable.

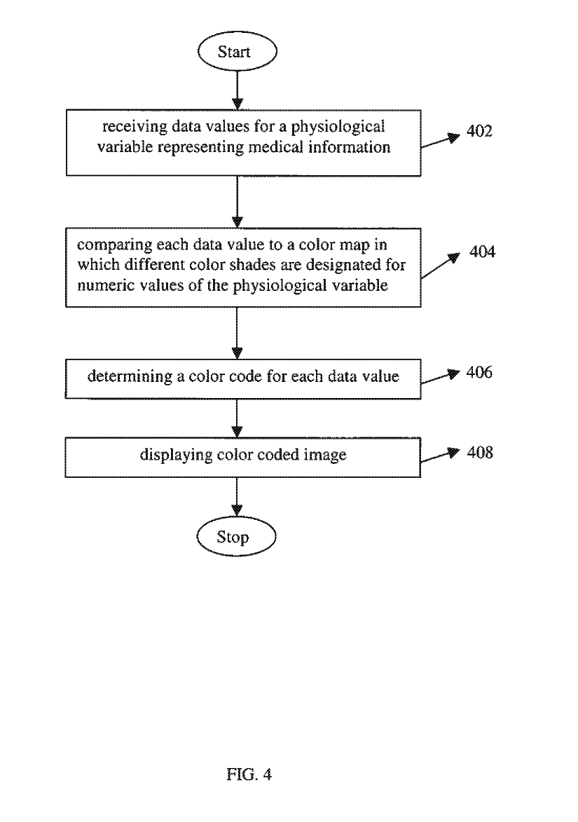

FIG. 4 is a flowchart showing processes that may occur with embodiments of the invention. Execution begins at step 402, where the medical information is obtained. The medical information is received numerically or graphically. The information can also be received electronically, such as utilizing a computer or electronic device, or can be transferred into an electronic format from a paper, dictation, or other format.

In the cases that the medical information has already been obtained. For example, previously existing information may be utilized. If no information need be obtained at the present time, execution proceeds to accessing existing information. Once the information has been accessed, the information may be converted into a color-coded format.

The medical information in the numerical form comprises set of data values for each physiological variable. At step 404 each of the data values for a selected physiological variable are compared to a color map to provide a determination of the color code for each data value. The color map may, for example, be a plurality of colored areas, such as squares in a grid with each square being a different color that represents a data value, determined by a scaling set by the user. Further, an individual color map may be provided for each physiological variable or a single color map may be used for all the physiological variables.

At step 406, in accordance with one embodiment, the color code for each physiological variable is generated from different color maps. For example, the color map employed for color-coding the data values of a first physiological variable may be different from the color map employed for color-coding the data values of a second physiological variable.

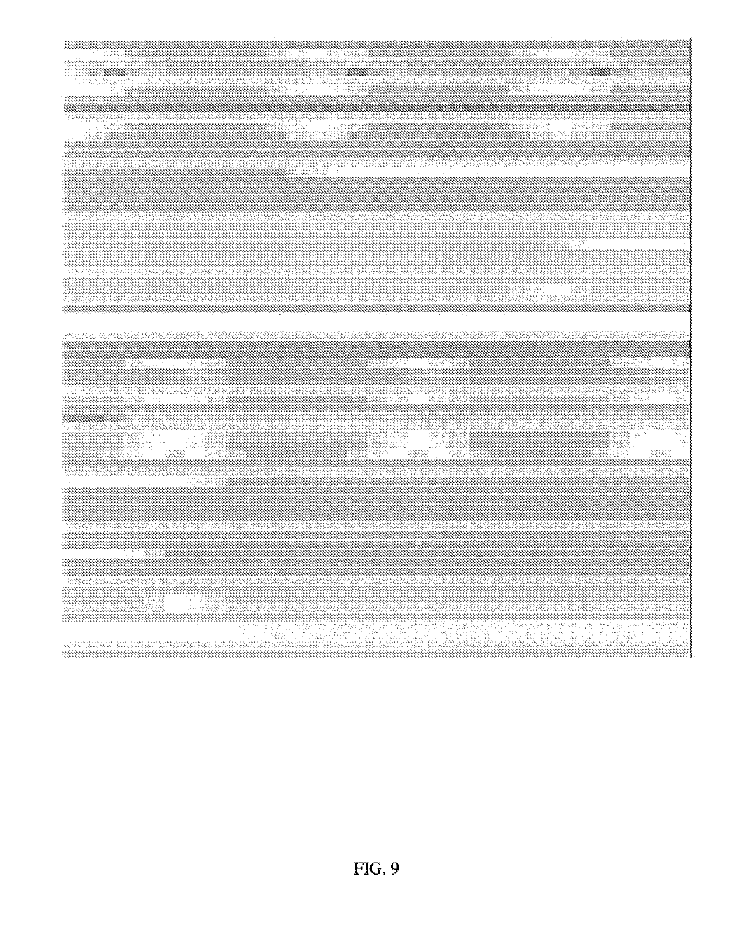

Alternatively, the color map may comprise a predetermined sequence of shades of an individual color. The color codes for the data values corresponding to a single physiological variable may be generated from the individual color. For this, each shade of the individual color is mapped to a single data value or a range of data values corresponding to the physiological variable. This is further explained in conjunction with FIG. 9.

Another method of illustrating the magnitude of data value is by using two colors. Similar to multi-color display one color is employed to indicate a maximum value and the other color is employed to indicate a minimum value. Further, shades of the colors are arranged to indicate intermediate values.

Once all the medical information is converted into color-coded image, execution may proceed to step 408 for displaying (either immediately or at a later time) the medical information.

As embodiments of the invention provide graphical representation of medical information, any type of display device or technology may be used in conjunction with embodiments of the invention, including conventional strip chart recorders, televisions, monitors, projectors, general-purpose and custom handheld screens, etc. It will be appreciated that future computer and graphical display technology may also be utilized with embodiments of the invention.

While the medical information may be obtained in non-graphical ways as mentioned in the above-described embodiments and then converted into a color-coded format, the medical information may also be obtained graphically.

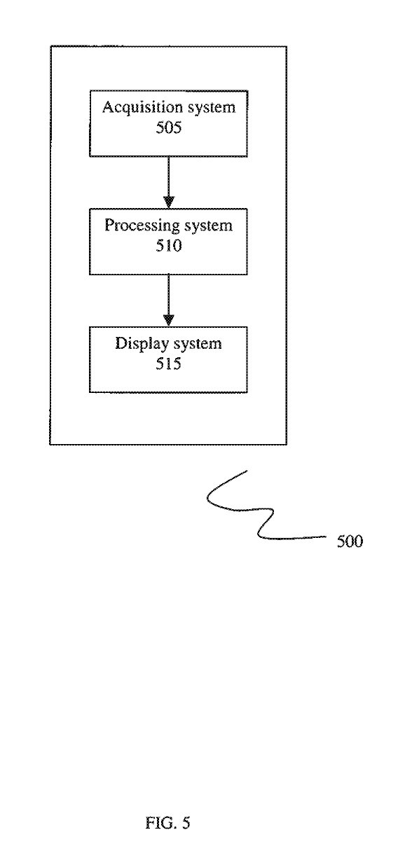

Accordingly, in yet another embodiment, a system for simultaneously displaying a plurality of physiological variables indicative of the condition of a patient is provided. The system 500 as shown in FIG. 5 comprises an acquisition system 505 for receiving medical information indicative of multiple categories, each category of medical information comprising signals corresponding to one or more physiological variables, a processing system 510 for generating a plurality of data values, each of which is indicative of one of the signals, the processing system further configured for converting each of the data values into color codes, and a display system 515 for separately and simultaneously displaying each of the color coded values in a graphical form in a time correlated relation wherein variations in the data values are emphasized by differences in color or shade.

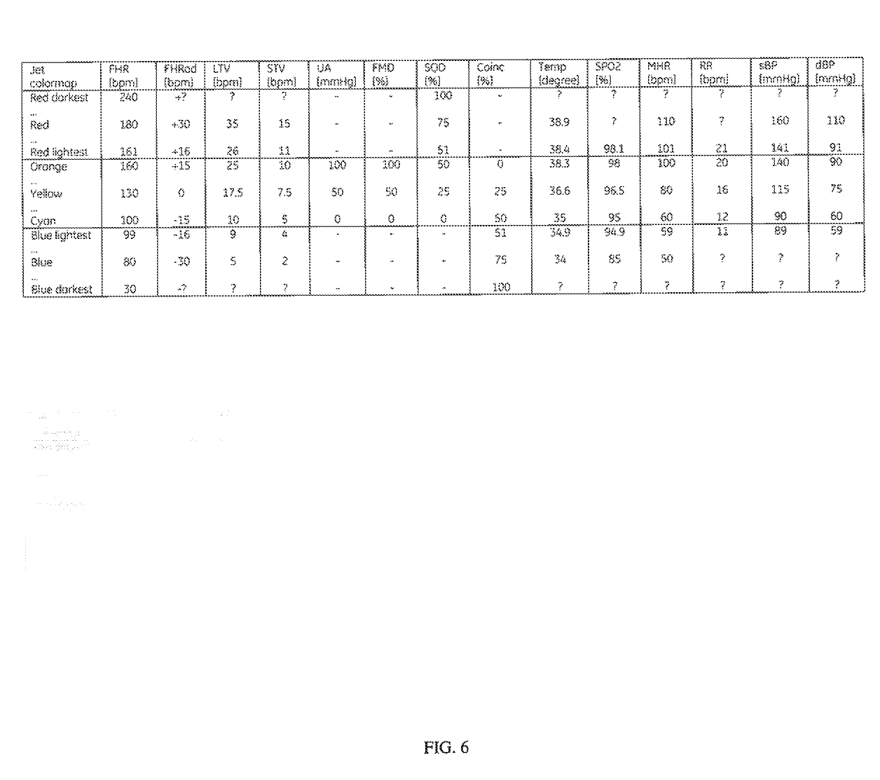

Color coded images (also referred to herein as colorgram) of medical information comprises many sections. Each section displays color codes corresponding to a single physiological variable. For example, one of the sections comprises colorgram for FHR waveform that represents a color-coded image of FHR waveform. To generate a colorgram each sample (waveform or signal) from the FHR waveform is converted into an indicator. The indicator comprises one of a bar, line, point or pixel in graphical form filled, bordered or marked with the mapped color. The color of the indicator for each sample is selected from a color table shown at FIG. 6.

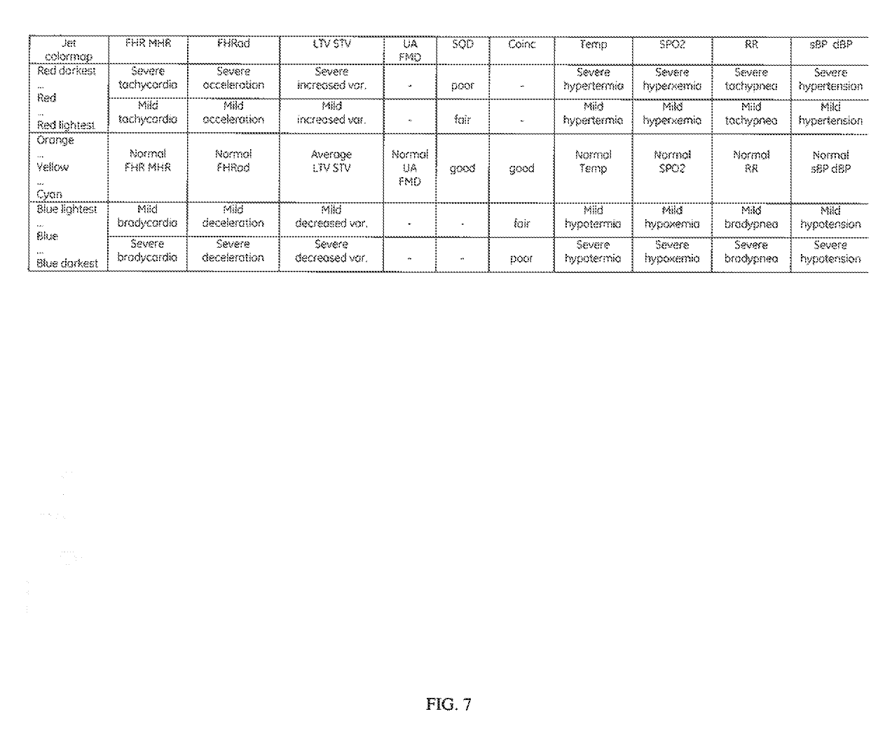

For example, in the colorgram for FHR waveform, presence of dark blue segment indicates the possibility of fetus suffering from severe bradycardia, presence of cyan, yellow or orange tints indicates that the FHR is normal and presence of light red segment indicates the possibility of fetus suffering from mild tachycardia. Table shown in FIG. 7 provides clinical meaning for the colors mapped for each physiological variable using jet colormap (shown in FIG. 2).

In one embodiment, the parameters for indicating the colorgram include type and name of waveform, screen resolution that depends on speed of the recorder, for example, a speed of 1 cm/minute indicates screen resolution of 40 pxl/minute, a speed of 2 cm/minute indicates screen resolution of 80 pxl/minute and so on, indicator size indicating the size of the indicator in terms of pixels 1*1 pixels, 2*2 pixels, 3*3 pixels etc. and window size representing the size of the window allocated for colorgram display.

Type and name of the waveform indicates the categories that may include fetal vital signs, maternal vital signs, integrated vital signs, medications taken by the patient and physical procedures applied to the patient during labor and delivery.

In the medications taken category each medication taken by the patient relates to a colorgram. The parameters for the medication waveform include dose, a first point of time to start taking medication, a second point of time to end taking medication and medication name.

In the physical procedure category each physical procedure applied to the patient relates to a colorgram. The parameters for the physical procedure waveform include intensity of physical procedure, a first point of time to start procedure, a second point of time to end procedure and procedure name.

The integrated vital signs category comprises arithmetic integration, logical integration, spatial integration and temporal integration. The arithmetic integration represents the arithmetic addition of selected physiological variables in the fetal vital signs and the maternal vital signs category. The arithmetic integration may be influenced by the weights distributed among the vital signs based on the significance given to the vital signs involved.

The logical integration is implemented by generating a digital output signal from an integration carried out and determined from the logical states of the physiological variables belonging to each one of the maternal vital signs and the fetal vital signs category.

The temporal integration combines vectors of the physiological variables belonging to the maternal vital signs and the fetal vital signs category. The parameters for temporal integration include vectors of vital signs and refresh rate. In one exemplary embodiment, the vector consists of FHR and UA waveforms and the refresh rate is selected to be 1 Hz. Accordingly, in the colorgram window, FHR colorgram is displayed for 1 second and the UA colorgram is displayed for 1 second, and the cycle is repeated.

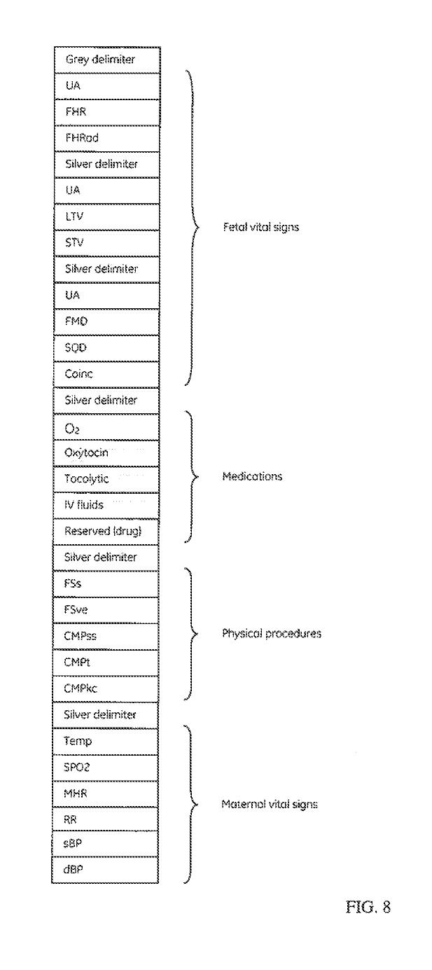

The spatial integration is based on the term macrocell. The macrocell is a combination of cells represented by vital signs, medication waveforms, physical procedure waveforms and cells-delimiters. Delimiters are cells with colors beyond the vital signs palette. Parameters for spatial integration include macrocell layout and macrocell size representing columns and rows. In one exemplary embodiment, a colorgram with macrocell 1[column]*34[rows] consisting of 34 vertical cells is shown in FIG. 8. The spatial integration can be used to provide the entire clinical picture in a condensed colorful form.

In one exemplary embodiment, FIG. 9 demonstrates a colorgram with the macrocell shown in FIG. 8. FIG. 9 comprises two groups, a first group and a second group, of 34 rows each. The second group shown below the first group is a continuation of the first group. Two rows of grey delimiters separate the groups.

The first group shows, in the fetal vital signs category the following physiological variables. UA: three contractions, FHR: three drops in baseline FHR, FHRad: three delayed mild decelerations, LTV: normal, STV: mild decreased variability, FMD: fetal movements coinciding with contractions, SQD: quality of UA signal normal, Coincidence: no coincidence of MHR and FHR

In the medications category, during second contraction oxygen is given to the patient to improve STV, however other medications are not used.

In the physical procedures category during third contraction maternal position is changed side-to-side to improve STV, however other procedures are not applied

In the maternal vital signs category the following physiological variables are displayed, Temp: normal, SPO2: mild hyperxemia after third contraction, MHR: mild tachycardia, RR: normal, sBP and dBP: blood pressure: normal but high.

The second group shows the effectiveness of oxygenation and physical procedure applied during the first half. In the fetal vital signs category, the following physiological variables are displayed. UA: three contractions, FHR: three mild drops in baseline, FHRad: three mild decelerations, LTV: normal, STV: mild decreased variability (indicating the effectiveness of oxygenation or application of physical procedure), FMD: fetal movements coincide with contractions, SQD: quality of UA signal is normal and becomes fair during contractions, Coincidence: no coincidence of MHR and FHR.

In the medications category, upon observing improvement in STV, oxygenation is stopped. Other medications are not used.

In the physical procedures category, upon observing improvement in STV the change of position side-to-side is stopped. Other procedures are not applied.

In the maternal vital signs category the following physiological variables are displayed. Temp: normal, SPO2: returns back to normal following oxygenation termination, MHR: back to normal, RR: normal, sBP and dBP: improved blood pressure.

The colorgram in FIG. 9 is shown as comprising shades of grey. Those skilled in the art will appreciate that the illustrated color codes are merely meant as examples. With the system according to the invention, the user can select or develop any color code, which illustrates variations in the data value. In addition, the amplitude scale of any of the individual displays may also be modified by the user.

Further, the user can add other physiological variables and delete some or all of those shown. Also, any variable can be moved, hidden or displayed at the user's discretion. As a result, the user can view as many or as few physiological variables at any given time as may be appropriate.

In one embodiment, the methods described herein may be employed to generate instructions for processor in maternal fetal monitor in order to implement color-coded cardiotocography. Accordingly, a computer program product for implementing within a computer system a method for representing patient medical information as a color gram is provided. The computer program product comprises a computer readable medium for providing computer program code means utilized to implement the method. The computer program code means comprises executable code for implementing steps for receiving one or more data values for a physiological variable representing medical information, comparing each data value to a color map in which different color shades are designated for numeric values of the physiological variable, determining a color code for each data value, generating a color coded image comprising a sequence of color codes representing various data values of the physiological variable and displaying the generated color coded image.

Color mapping of a physiological variable is done by assigning a particular color to a particular data value or range of data values in such a manner that changes in color are related to concomitant changes in the data value. The colors are then embedded into an indicator thereby allowing simultaneous display of color-coded images of various physiological variables. It will be appreciated that such a representation of colors may permit more medical information to be displayed on a single graphical display, as the indicators in such embodiments do not rely on physical size to convey the medical information. The color-coded medical information thus displayed can be quickly and rapidly viewed and digested.

It should be appreciated that in some instances it may be desirable to provide more detailed information than can be graphically represented in a single graphical representation. In some embodiments, additional information may be presented textually or otherwise.

In some embodiments, the graphical and textual information may be simultaneously presented, such as side-by-side or above-and-below, to best convey information in the manner desired by the user. Additionally, the “textual information” displayed may include graphical or other information of the type currently provided with patient medical information, where appropriate. In this way, the user may be provided with all the information available previously, but supplemented with the rapidly digested color-coded information discussed herein.

As may be appreciated, a user viewing the color-coded image may very quickly be able to determine areas of potential trouble for the associated patient. Indeed, as the user becomes familiar with the information displayed in the color-coded representation, it will become less necessary for the user to refer to the category descriptions that may be displayed on the graphical representation.

For example, a medical professional may begin to recognize and associate certain color-coded patterns with certain diagnoses. As an example, a medical professional may recognize that a certain pattern or set of colors in the FHR physiological variable is associated with severe bradycardia. Upon later encountering a similar pattern the medical professional may suspect bradycardia. In this way, embodiments of the invention might assist with diagnosis, even in earlier stages than might otherwise be caught.

In some embodiments it may be possible to include software-based or other semi-automatic or automatic recognition of certain graphical patterns to provide tentative or potential diagnosis information to the user. In embodiments where computer programs are utilized, the computer programs may include diagnosis information based on patterns discovered over tens, hundreds, or thousands of patients, along with the associated diagnoses. This information may be displayed with the color-coded representations and/or separately.

The above descriptions of possible uses and applications of the color-coded images of medical information are considered illustrative only. Additional uses and applications may be learned by the practice of the embodiments of the invention. Additionally, the illustrated graphical representations are intended to be representative only.

The embodiments of the invention described herein provide a new approach to CTG (Cardiotocography) referred to as ccCTG (color-coded CTG). ccCTG allows medical professionals to think of clinical picture in terms of colorgrams rather than conventional waveforms or numbers. It may also be appreciated that, through practice, the medical professional may easily differentiate between a normal clinical condition and a pathological clinical condition as a reassuring clinical condition has different coloration relatively to a nonreassuring one.

A user of the embodiments of the invention will be able to nearly instantly determine problem and/or non-problem areas utilizing the color-coded representations discussed herein. Therefore the problem of misinterpretation of fetal tracings peculiar to conventional CTG is likely to be resolved in the color-coded representation.

Color coded image representation of medical information covers clinical picture spanning a longer time duration. For example, 21 cm of conventional strip chart recording at speed 3 cm/minute contains only seven minutes of FHR and UA waveforms whereas the same area of color-coded cardiotocogram at the same speed contains medical information spanning approximately two hours. Further, additional information such as fetal vital signs, maternal vital signs, medications taken by patient and physical procedures applied to the patient can as well be displayed. As a result, a user may obtain more informative clinical picture spanning a longer duration in a single view.

Thus, instead of having to wade through a long recording of the strip chart, the user can obtain what is likely to be most relevant and important medical information at a glance.

In various embodiments of the invention, systems and methods for conveying medical information in a color-coded format are described. However, the embodiments are not limited and may be implemented in connection with different applications. The application of the invention can be extended to other areas, for example in the area of providing dietary decisions, fitness evaluation, and other types of care, diagnosis and treatments to an individual. The invention provides a broad concept of monitoring multiple continuously varying input variables in order to maintain optimum conditions in a system or to respond to or anticipate emergency situations, which can be adapted in a similar monitoring system. Accordingly, while the invention is illustrated in connection with a medical monitor, it may employed for monitoring the operation of other complicated systems with a large number of variables, such as, aircraft, flight control tower systems, nuclear power plants, electrical grids, and complex industrial manufacturing or processing operations.

This written description uses examples to describe the subject matter herein, including the best mode, and also to enable any person skilled in the art to make and use the subject matter. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An apparatus for graphical display of medical information, the apparatus comprising:
a memory configured to store medical information indicative of multiple categories, each category of medical information representing information about plurality of physiological variables related to patient monitoring;
a processing system configured to determine a data value for at least one physiological variable in each of the category of medical information and further configured for generating a color code using the data values; and
a display system for displaying a color coded image comprising multiple sections, each section displaying color codes associated with one of the physiological variables.

2. The apparatus of claim 1, wherein the color code is generated from a color map, the color map comprising a predetermined sequence of colors selected from visible color spectrum consisting of the colors red, orange, yellow, green, blue, indigo and violet and wherein each color is mapped to a single data value or a range of data values corresponding to a single physiological variable.

3. The apparatus of claim 2, wherein the color code for each physiological variable is generated from different color maps.

4. The apparatus of claim 1, wherein the color code is generated from a color map, the color map comprising a predetermined sequence of shades of a single color and wherein each shade is mapped to a single data value or a range of data values corresponding to a single physiological variable.

5. The apparatus of claim 1, wherein each category of medical information comprises one of fetal vital signs, maternal vital signs, integrated vital signs, medication and physical procedure.

6. The apparatus of claim 5, wherein the fetal vital signs category comprises one of a fetal heart rate (FHR) waveform, fetal heart rate acceleration or deceleration waveform, long term FHR variability waveform, short term FHR variability waveform, uterine activity waveform, fetal movement detection waveform, signal quality detection waveform and coincidence waveform.

7. The apparatus of claim 5, wherein the maternal vital signs category comprises one of a temperature waveform, pulse oximeter oxygen saturation waveform, maternal heart rate waveform, respiration rate waveform, systolic blood pressure waveform and diastolic blood pressure waveform.

8. The apparatus of claim 5, wherein the integrated vital signs category comprises one of an arithmetic integration waveform, logical integration waveform, temporal integration waveform and spatial integration waveform.

9. The apparatus of claim 5, wherein the medication category comprises one of a dose, first point of time indicating a start time for taking medication, second point of time indicating an end time for taking medication and medication name.

10. The apparatus of claim 5, wherein the physical procedure category comprises one of a fetal stimulus by sound waveform, fetal stimulus by vaginal exam waveform, change maternal position side-to-side waveform, change maternal position Trendlenburg waveform and change maternal position knee-chest waveform.

11. A system for simultaneously displaying a plurality of physiological variables indicative of the condition of a patient, the system comprising:
an acquisition system for receiving medical information indicative of multiple categories, each category of medical information comprising signals corresponding to one or more physiological variables related to patient monitoring;
a processing system for generating plurality of data values each of which is indicative of one of the signals, the processing system further configured for converting each of the data values into color codes; and
a display system for separately and simultaneously displaying each of the color coded values in a graphical form in a time correlated relation wherein variations in the data values are emphasized by differences in color or shade.

12. The systems of claim 11, wherein the color codes for the data values corresponding to a single physiological variable are generated from a color map, the color map representing a predetermined sequence of colors selected from visible spectrum consisting of the colors red, orange, yellow, green, blue, indigo and violet.

13. The system of claim 12, wherein the color map employed for color coding the data values of a first physiological variable is different from the color map employed for color coding the data values of a second physiological variable.

14. The system of claim 11, wherein the color codes for the data values corresponding to a single physiological variable are generated from an individual color and wherein the amplitude of the physiological variable determines the shade of color.

15. A method for generating color coded image of medical information, the method comprising:
obtaining one or more data values for a physiological variable representing medical information;
grouping the data values into one or more data ranges, the data range representing a range of data values;
mapping colors to data ranges in a manner that allows the colors to visually indicate the magnitude of the data ranges; and
marking an indicator for each data value on a graph, the indicator marked with the mapped color of the data range that comprises the data value of the physiological variable.

16. The method of claim 15, wherein the indicator comprises one of a bar, line, point or pixel in graphical form filled, bordered or marked with the mapped color that has been mapped to the data range to which the data value of the physiological variable belongs.

17. A computer program product for implementing within a computer system a method for representing patient medical information as a colorgram, the computer program product comprising: a computer readable medium for providing computer program code means utilized to implement the method, wherein the computer program code means is comprised of executable code for implementing steps for receiving one or more data values for a physiological variable representing medical information, comparing each data value to a color map in which different color shades are designated for numeric values of the physiological variable, determining a color code for each data value and displaying color coded image, the color coded image comprising a sequence of color codes representing various data values of the physiological variable.

18. The computer program product of claim 20, further comprising executable code for displaying changes in amplitude of the data values corresponding to a single physiological variable as varying shades of a single color.
